(19) United States
(12) Patent Application Publication
(10) Pub. No.: US 2018/0271441 A1
(43) Pub. Date: Sep. 27, 2018

(54) WEARABLE ELECTRODE AND METHOD OF FABRICATION

(71) Applicant: Intel Corporation, Santa Clara, CA (US)

(72) Inventor: Nadine L. Dabby, Palo Alto, CA (US)

(21) Appl. No.: 15/467,235

(22) Filed: Mar. 23, 2017

(51) Int. Cl. A61B 5/00; H01B 5/14; H01B 13/00; A41D 1/00; A61B 5/04

(52) U.S. Cl. CPC A61B 5/6804; H01B 5/14; H01B 13/0036; A61B 2560/0468; A61B 5/04; A61B 2562/125; A41D 1/002

(57) ABSTRACT

A wearable electrode includes a first layer of a first material, a second material positioned on the first material, the second material having a first compressive strength, a third material positioned on the second material, the third material having a second compressive strength different than the first compressive strength and a fourth material including a conductive element positioned on the third material, positioned around the second material, and joined to the first material.

### FIELD OF THE DISCLOSURE

This disclosure relates generally to wearables and wearable devices.

### BACKGROUND

A variety of wearable devices include one or more electrodes. In some examples, wearables include a conductive fabric (e.g., a fabric having fibers coated with a conductive material, such as silver, or a conductive ink, etc.). These conductive fibers may be used to operatively connect an electrode or sensor on an inner, skin-facing side of the wearable to a device, such as a heart rate monitor, on an outside of the wearable device.

### BRIEF DESCRIPTION OF THE DRAWINGS

FIG. 1 depicts a first example wearable having example wearable electrodes constructed in accordance with teachings of this disclosure.

FIG. 2 depicts a second example wearable having example wearable electrodes constructed in accordance with teachings of this disclosure.

FIGS. 3A-3H depict example fabrication stages of example wearable electrodes in accordance with teachings of this disclosure, with FIG. 3H depicting a cross-section taken along line 3H-3H of FIG. 2.

FIG. 4 is a block diagram of an example implementation of wearable electrode former to perform the fabrication stages of FIGS. 3A-3H to form the example wearables of FIGS. 1-2 in accordance with teachings of this disclosure.

FIGS. 5A-5B are block diagrams of example apparatus that can be used by the example wearable electrode former of FIG. 4 to fabricate the example wearables having the example wearable electrodes of FIGS. 1-2 in accordance with teachings of this disclosure.

FIGS. 6A-6B are flowcharts representative of machine readable instructions which may be executed to implement the example wearable electrode former of FIG. 4, the example apparatus shown in FIGS. 5A-5B, the example fabrication of FIGS. 3A-3H and/or the example wearables having the example wearable electrodes of FIGS. 1-2 in accordance with teachings of this disclosure.

FIG. 7 is a block diagram of an example processor platform which may execute the example instructions of FIGS. 6A-6B to implement the example wearable electrode former of FIG. 4, the example apparatus shown in FIGS. 5A-5B, the example fabrication of FIGS. 3A-3H and/or the example wearables having the example wearable electrodes of FIGS. 1-2.

The figures are not to scale. Instead, to clarify multiple layers and regions, the thickness of the layers may be enlarged in the drawings. Wherever possible, the same reference numbers will be used throughout the drawing(s) and accompanying written description to refer to the same or like parts. As used in this patent, stating that any part or material (e.g., a layer, film, area, or plate) is in any way positioned on (e.g., positioned on, located on, disposed on, or formed on, etc.) another part or material, means that the referenced part is either in contact with the other part, or that the referenced part is spaced apart from another part or material with one or more intermediate part(s) or material(s) intervening therebetween. Stating that any part is in contact with another part means that there is no intermediate part between the two parts.

### DETAILED DESCRIPTION

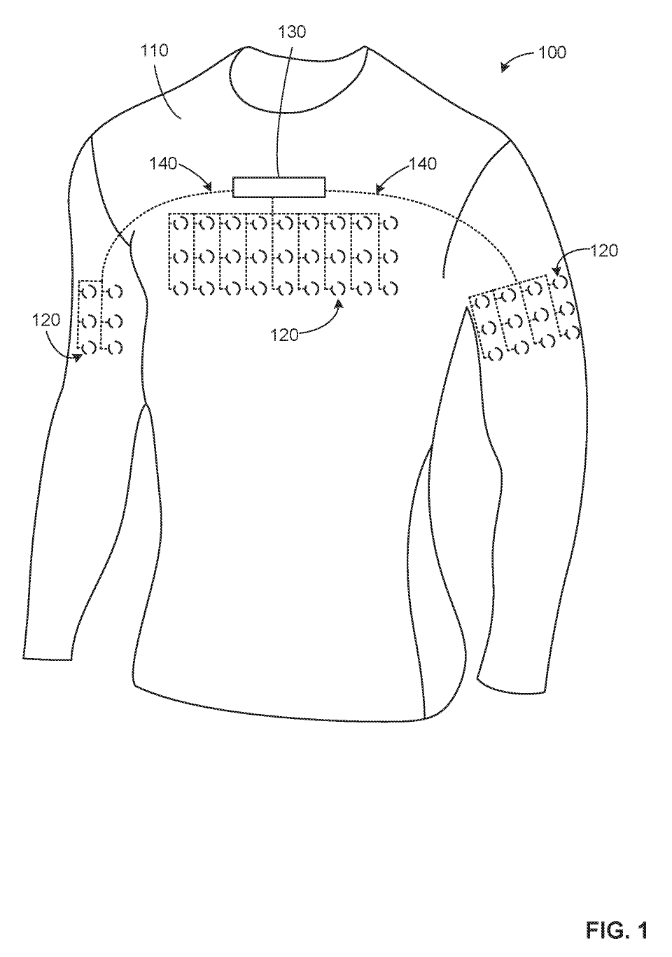

FIG. 1 depicts an example wearable 100 in accordance with teachings of this disclosure. The example wearable 100 shown in FIG. 1 is activewear 110, more particularly a long-sleeve sports shirt, having example wearable electrodes 120 distributed about the upper arms and the chest of the example activewear 110. In other examples, the activewear 110 could include a bra, shorts, a pant, a sock, a compression garment, or any other garment in contact with the skin. The example wearable electrodes 120 provide data relating to a user (e.g., a wearer, etc.) of the activewear 110 to a local device 130 via electrical pathways 140. The data may include, for example, biometric data, heart rate data, respiration rate data, breathing depth data, power, acceleration, speed, reps, calories burned data and/or steps taken data. The local device 130 may include, for example, a 3-axis accelerometer, a clock, a global positioning system (GPS) device, a transceiver (e.g., Bluetooth, ultra-wideband (UWB), Wi-Fi, ZigBee, Radio frequency (RF) in the 863-870 MHz, 902-928 MHz, 2.4-2.5 GHz or 5.7-5.8 GHz bands, etc.), a battery, a light emitting diode (LED)(e.g., a photoplethysmography sensor, etc.), a thermometer and/or a bioimpedance sensor.

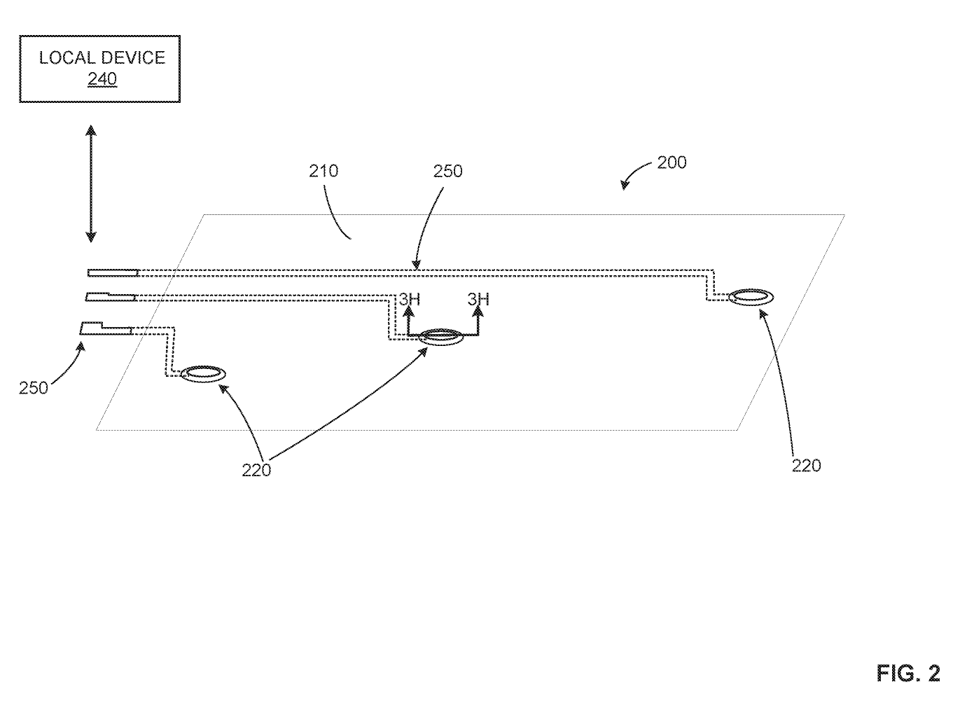

FIG. 2 depicts another example wearable 200 in accordance with teachings of this disclosure. The example wearable 200 shown in FIG. 2 is a wearable material 210 (e.g., a strip of material) including one or more wearable electrodes 220 that may be adapted to suit different wearable form factors (e.g., a strap of a bra, a strap of a short, a compression wrap, a head band, a wrist band, etc.) and different needs (e.g., a wellness/fitness device, a medical device, etc.). The example of FIG. 2 depicts a staggered arrangement of wearable electrodes 220 distributed along a length of the wearable material 210. In other examples, the wearable material 210 includes one or more wearable electrodes 220 in any regular or irregular arrangement or in any pattern (e.g., an array, etc.).

The example wearable electrodes 220 of the wearable material 210 of FIG. 2 provide, during use, data relating to a user (e.g., a patient, a wearer, etc.) of the wearable material 210 to a local device 240 connected to the electrical pathways 250 via a hardwired connection and/or a wireless connection (e.g., Bluetooth, RF, etc.). The local device 240 may include, for example, for a material 210 implemented in a medical device, a vital signs monitor (e.g., temperature, pulse rate, respiration rate, etc.), an anesthesia machine, a pulse oximeter, an electrocardiogram (EKG/ECG) machine, an electromyography (EMG) machine, and/or an electroencephalography (EEG) machine. The data may include, for example, biometric, EMG, galvanic skin response (GSR) and/or EKG/ECG data.

In FIG. 2, the example material 210 is shown with an inner surface (e.g., a surface to be placed against the skin of a user) facing upwardly. The example wearable electrodes 220 project outwardly (e.g., upwardly as shown in FIG. 2) from the inner surface of the wearable 200 adjacent the skin of the user. Likewise, the example wearable electrodes 120 of the wearable 100 of FIG. 1 project outwardly (e.g., inwardly as shown in FIG. 1) from the inner surface of the wearable 100, toward the skin of the user. The projection of the example wearable electrodes 120, 220 from an inner surface of the example wearables 100, 200 facilitates contact between the wearable electrodes 120, 220 and the skin of the user and avoids the need to impart force to press the wearable electrodes 120, 220 into the skin, as is required by some types of biometric devices which utilize an elastic strap to bias a hard housing (e.g., polypropylene, high-density polyethylene, etc.) bearing an electrode against a user's skin. The projection of the example wearable electrodes 120, 220 from the surface of the example wearables 100, 200 also avoids issues of the poor or inadequate contact exhibited by fabric-based electrodes reliant on conductive inks or conductive fabric. As discussed below, the wearable electrodes 120, 220 are, in some examples, textile-based electrodes including two or more layers of materials with at least two different compressive strengths between a first flexible material (e.g., an outer layer of a wearable 100, 200) and a second flexible material (e.g., an inner layer of a wearable 100, 200) including a conductive fabric.

The use of two or more layers of materials with at least two different compressive strengths, particularly if arranged with a material having a lower compressive strength closer to an inner layer of a garment or a wearable (e.g., closer to the skin of a user) and a material with a higher compressive strength closer to an outer layer of the wearable (e.g., further from the skin of the user), improves fit and contact between the wearable electrodes 120, 220 and the user's skin by permitting the more malleable material (i.e., the material having the lower compressive strength) to better conform to a local topography of the user's skin.

Although the example wearable electrodes 120, 220 are shown to be generally circular, by way of example in FIGS. 1-2, the form factor and/or size of the electrode may include any symmetric shape (e.g., circular, square, rectangular, oval, etc.), asymmetric shape (e.g., semi-circle, etc.) and/or size, without limitation.

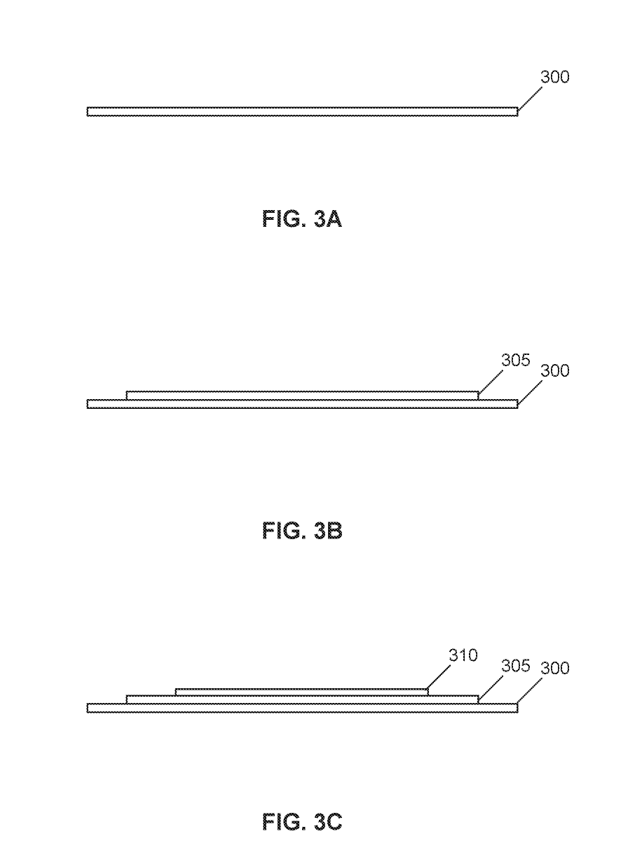

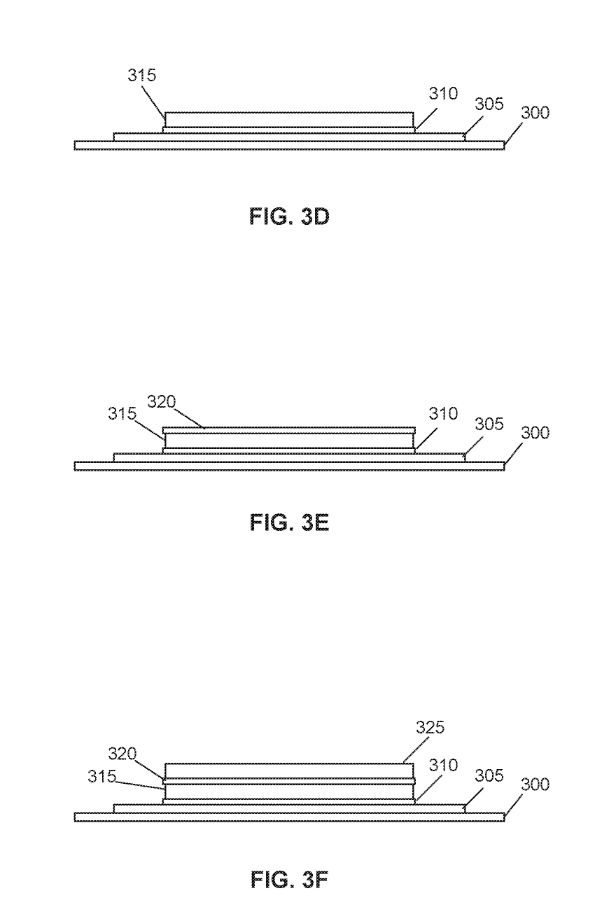

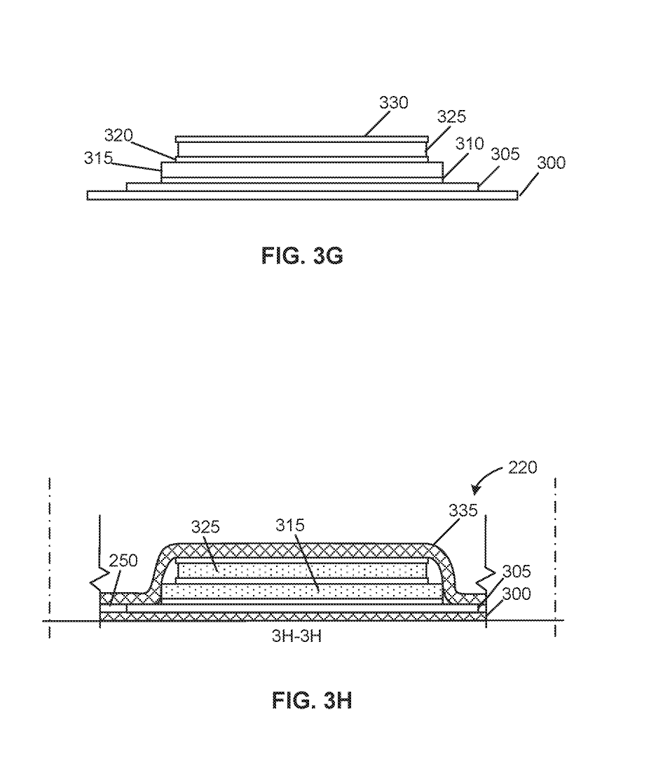

FIGS. 3A-3H depict various fabrication stages of the example wearable electrodes 120, 220 for a wearable 100, 200 in accordance with teachings of this disclosure.

FIG. 3A shows the positioning of a flexible first material 300 as a base layer of a workpiece upon which one or more example wearable electrodes 120, 220 are formed. In an example of a wearable 100, such as activewear, the first material 300 includes a flexible material appropriate to the activewear (e.g., selected based on intended use and environment(s) in which the activewear is used, etc.). In some examples, the first material 300 may include a non-cellulose natural material (e.g., wool, silk, leather, etc.), a cellulose material (e.g., cotton, recycled paper, linen, bamboo, jute, hemp, flax, etc.) and/or a synthetic material (e.g., nylon, polyester, spandex, elastane, neoprene, acetate, Orlon, Kevlar, latex, rayon, polytetrafluoroethylene (PTFE), etc.). In one example, a wearable is an activewear 110, such as shown in FIG. 1, including a first material 300 that is 92% polyester and 8% spandex. In some examples, the wearable 100, 200 includes a synthetic stretch fabric (e.g., a 2-way stretch fabric, a 4-way stretch fabric, etc.).

In the example of FIG. 3B, a waterproof barrier material 305 has been positioned on the first material 300. As noted above, the positioning of the waterproof barrier material 305 on the first material 300 may mean that the waterproof barrier material 305 is in contact with the first material 300 or that the waterproof barrier material 305 is positioned on the first material 300 with one or more intermediate part(s) or material(s) intervening therebetween. In some examples, the waterproof barrier material 305 includes a polyurethane laminate of polyester fabric and a thermoplastic polyurethane (TPU), an expanded polytetrafluoroethylene (ePTFE), a laminate of an ePTFE and a polyurethane (PU), a liquid coating such as an ultra-hydrophobic or a superhydrophobic coating or a nanocoating. In some examples, the waterproof barrier material 305 is bonded to the first material 300, by application of heat and/or pressure for a predetermined time appropriate to the selected adhesive. For example, the workpiece of FIG. 3B including the first material 300 and the waterproof barrier material 305 is heated to a temperature of about 125° C. for about 30 seconds via a heat press or mold. In other examples, the waterproof barrier material 305 is mechanically bonded, chemically bonded, or thermally bonded, or joined by stitching. In some examples, the example waterproof barrier material 305 is omitted. For instance, the first material 300 includes a waterproof material, coating, or treatment, rendering application of a waterproof barrier material 305 superfluous.

FIG. 3C shows positioning of a first bonding material 310 on the waterproof barrier material 305. The first bonding material 310 may be in contact with the waterproof barrier material 305 or the first bonding material 310 may be positioned on the waterproof barrier material 305 with one or more intermediate part(s) or material(s) intervening therebetween. In other examples, the first bonding material 310 may be in contact with the first material 300 (e.g., the waterproof barrier material 305 may be omitted, the first bonding material 310 may cover the waterproof barrier material 305 and contact the first material as well, etc.). In some examples, the first bonding material 310 is a wet adhesive (e.g., a liquid, an aqueous-based adhesive, a solvent-based adhesive, a spray-on fabric adhesive, etc.) or a dry adhesive (e.g., a fiber, a powder, a paste, a hot-melt thermoplastic adhesive, a bond tape, etc.). In some examples, the first bonding material 310 may include a double-sided fabric tape (e.g., Aleene's Fabric Fusion Sheets, Bemis tapes, etc.). In some examples, the first bonding material 310 may include a butadiene polymer, an acrylic acid or saturated polymer, a vinyl polymer (e.g., vinyl acetate, vinyl ether, vinyl ester, vinyl chloride, etc.), thermoplastic polymer bonding fibers (e.g., polyvinyl alcohol (PVA), co-polyamide, polyolefin, polyester and polyvinylchloride, an ester polyurethane or a paste or powder including co-polyamides, polyethylene, or ethylene vinyl acetate (EVA) copolymers.

In some examples, the first bonding material 310 is bonded to the waterproof barrier material 305 by application of heat and/or pressure (e.g., a hot melt lamination, a flame lamination, application of a heat press or mold, etc.) for a predetermined time appropriate to the selected adhesive. For example, the workpiece of FIG. 3C including the first material 300, the waterproof barrier material 305 and the first bonding material 310 is heated to a temperature of about 125° C. for about 30 seconds via a heat press or mold. In some examples, the first bonding material 310 (e.g., a thermo-set adhesive, etc.) is positioned on the waterproof barrier material 305 in a discontinuous manner on one or more discreet locations (e.g., a dot or a pattern of dots, etc.).

FIG. 3D shows positioning of a second material 315 on the first bonding material 310 of the workpiece shown in FIG. 3C. In some examples, the second material 315 is in contact with the first bonding material 310. In some examples, the second material 315 is positioned on the first bonding material 310 with one or more intermediate part(s) or material(s) intervening therebetween. In other examples, the second material 315 is in contact with the first material 300 (e.g., the waterproof barrier material 305 and the first bonding material 310 may be omitted, etc.). The second material 315 has a first compressive strength. In some examples, the first compressive strength is between about 10-40 pounds per square inch (PSI). In some examples, the second material is a foam material such as, for example, a microcellular urethane, a polyurethane, an expanded polystyrene (EPS), an extruded polystyrene (XPS), or a polyethylene.

In some examples, following positioning of the second material 315 as shown in FIG. 3D, the second material 315 is bonded to the first bonding material 310, or is bonded to the first material 300 depending on configuration, by application of heat and/or pressure (e.g., a hot melt lamination, a flame lamination, application of a heat press or mold, etc.) for a predetermined time appropriate to the selected bonding material. For example, the workpiece of FIG. 3D including the first material 300, the waterproof barrier material 305, the first bonding material 310 and the second material 315 is heated to a temperature of about 125° C. for about 30 seconds via a heat press or mold.

FIG. 3E shows positioning of a second bonding material 320 on the second material 315 of the workpiece shown in FIG. 3D. The second bonding material 320 may be in contact with the second material 315 or the second bonding material 320 may be positioned on the second material 315 with one or more intermediate part(s) or material(s) intervening therebetween. In other examples, the second bonding material 320 may be omitted. In some examples, the second bonding material 320 may be the same material as the first bonding material 310 or may be a different bonding material. In some examples, the second bonding material 320 may be positioned or applied in the same manner as the first bonding material 310 is applied, or it may be positioned or applied in a different manner.

FIG. 3F shows positioning of a third material 325 on the second bonding material 320 of the workpiece shown in FIG. 3E. In some examples, the third material 325 is in contact with the second bonding material 320. In some examples, the third material 325 is positioned on the second bonding material 320 with one or more intermediate part(s) or material(s) intervening therebetween. In other examples, the third material 325 is in contact with the second material 315 (e.g., the second bonding material 320 is omitted, etc.). The third material 325 has a second compressive strength different than that of the first compressive strength of the second material 315. In some examples, the second compressive strength is between about 1-10 PSI. In some examples, the third material 325 may be the same material as the second material 315 (e.g., the same material, but processed to have a different compressive strength via differences in physical processes (e.g., cutting, etching, etc.) or chemical processes (e.g., curing, etc.). In some examples, the third material 325 is a different material than the second material 315. In some examples, the third material 325 may be positioned on the second bonding material 320 or second material 315 in the same manner and/or a different manner as the second material 315. Following positioning of the third material 325 as shown in FIG. 3F, the third material 325 is bonded to the second bonding material 320, or is bonded to the second material 315 depending on configuration, by application of heat and/or pressure for a predetermined time appropriate to the selected bonding material. For example, the workpiece of FIG. 3F including the first material 300, the waterproof barrier material 305, the first bonding material 310, the second material 315, the second bonding material 320 and the third material 325, is heated to a temperature of about 125° C. for about 30 seconds via a heat press or mold.

FIG. 3F shows positioning of a third bonding material 330 on the third material 325 of the workpiece shown in FIG. 3E. The third bonding material 330 may be in contact with the third material 325 or the third bonding material 330 may be positioned on the third material 325 with one or more intermediate part(s) or material(s) intervening therebetween. In other examples, the third bonding material 330 may be omitted. In some examples, the third bonding material 330 may be the same material as the first bonding material 310 and/or second bonding material 320 or may be a different bonding material. In some examples, the third bonding material 330 may be positioned or applied in the same manner as the first bonding material 310 and/or the second bonding material 320 is applied, or it may be positioned or applied in a different manner.

FIG. 3H shows positioning of a fourth material 335 on the third bonding material 330 of the workpiece shown in FIG. 3G. In some examples, the fourth material 335 is a material that is the same as, or different from, the first material 300. In some examples, the fourth material 335 is a waterproof fabric or waterproof material. The fourth material 335 includes a conductive element to conduct electricity. In some examples, the fourth material 335 includes one or more embedded conductive elements (e.g., copper, silver, etc.). For instance, a textile material used as the fourth material 335 has a conductive wire wrapped around one or more threads of the textile material or a conductive coating applied to one or more threads of the textile material. In some examples, a conductive element is substantially uniformly distributed throughout the fourth material 335. As shown in the example of FIG. 3H, the fourth material 335, which is conductive, contacts the electrical pathway 250 to form a conductive path between the user and the example local device 240 to permit transmission of signals (e.g., heart rate, etc.) from the wearable electrode 220 to the local device 240.

While example fabrication stages are depicted in FIGS. 3A-3H, an order of some of the fabrication stages may be altered and/or some of the fabrication stages may be altered in manners known to those of ordinary skill in the art. For instance, in the fabrication stage depicted in FIG. 3C where an example first bonding material 310 is applied to an example waterproof barrier material 305, the example first bonding material 310 could instead be applied directly to the example second material 315 and bonded thereto, with the combination of the example first bonding material 310 and the example second material 315 then being applied to and bonded to the example waterproof barrier material 305.

In some examples, the fourth material 335 is a skin-facing inner layer of an electronic textile that may be incorporated into a wearable 100 (e.g., activewear, a garment, etc.) or a wearable 200 (e.g., a material 210, etc.), as shown by way of example in FIGS. 1-2. In some examples, a conductive element is positioned in the fourth material 335 to provide an electrical connection between a first location of the wearable electrode 120, 220 (e.g., a top portion of the wearable electrode 120, 220, where the conductive element is exposed to contact the skin of a user) and a second location of the wearable electrode 120, 220 (e.g., electrical pathways 140 embedded in the wearable 100 of FIG. 1, electrical pathways 250 embedded in the wearable 200, etc.). In some examples, the electrical pathways 140 include conventional wires, conductive thread, metal monofilaments, treated conductive fibers (e.g., coating fibers with metals or galvanic substances, etc.), conductive fabrics, conductive inks, metallizations or metallic films. In some examples, the electrical pathways 140 are stretchable. While, for simplicity, the electrical pathways 140 are represented in generally straight lines, the electrical pathways 140 may include other geometries, such as a serpentine pattern, to facilitate flexure and stretching.

In some examples, the fourth material 335 is positioned over the entirety of the first material 300. For instance, the fourth material 335 is an inner material of a wearable 200 (e.g., activewear 110 in FIG. 1) and the first material 300 is the outer material. In some examples, the fourth material 335 is positioned only over portions of the first material 300 which are to bear the wearable electrode(s) 220.

In some examples, the fourth material 335 is in contact with the third bonding material 330. In some examples, the fourth material 335 is positioned on the third bonding material 330 with one or more intermediate part(s) or material(s) intervening therebetween. In other examples, the fourth material 335 is in contact with the third material 325 (e.g., the third bonding material 330 is omitted, etc.). The fourth material 335 is not only positioned on the third material 325, but is also positioned around the second material 315, such as is shown in FIG. 3H.

In some examples, following positioning of the fourth material 335 as shown in FIG. 3H, the fourth material 335 is bonded to the third bonding material 330, the third material 325, the second material 315, the waterproof barrier material 305 and/or the first material 300, depending on configuration, by application of heat and/or pressure for a predetermined time appropriate to the selected bonding material to seal the fourth material 335 around the second material 315 and the third material 325 to form the example wearable electrode 220 on the first material. For example, the workpiece of FIG. 3H including the first material 300, the waterproof barrier material 305, the first bonding material 310, the second material 315, the second bonding material 320, the third material 325, the third bonding material 330 and the fourth material 335 is heated to a temperature of about 125° C. for about 30 seconds via a heat press or mold to bond the fourth material 335 to the first material 300, the waterproof barrier material 305, the second material 315 and/or the third material 325.

In some examples, the electrical pathway 250 is formed over the construct of the first material 300, the waterproof barrier material 305, the first bonding material 310, the second material 315, the second bonding material 320, the third material 325, and the third bonding material 330 before application of the fourth material 335. For example, the example fourth material 335 includes openings to expose the electrical pathway 250 to permit contact with a user's skin. In some examples, the example fourth material 335 is a non-conductive material (e.g., cotton, natural fibers, elastane, etc.) and conduction between a skin of the user and the local device(s) 130, 240 is via skin to electrical pathway 130, 250 contact through openings formed in the example fourth material 335.

Although the wearable electrode 220 is shown to have two layers of materials with at least two different compressive strengths (e.g., second material 315 and third material 325 in FIG. 3H), additional layers of materials having different compressive strengths may be used. For example, the wearable electrode 220 may have three layers of materials with at least two different compressive strengths (e.g., three different compressive strengths with a first material having a first compressive strength between about 20-40 PSI, a second material having a second compressive strength between about 10-20 PSI, and a third material having a third compressive strength between about 1-10 PSI). As another example, a wearable electrode 220 may have three layers of materials with three different compressive strengths, such as a first material having a first compressive strength between about 20-40 PSI, a second material having a second compressive strength between about 5-10 PSI, and a third material having a third compressive strength between about 1-5 PSI. In some examples, compressive strengths of the materials in wearable electrodes 120, 220, as well as layering of different numbers of layers in the wearable electrodes 120, 220, are individually tailored to specific regions and/or purposes of the wearable 100, 200.

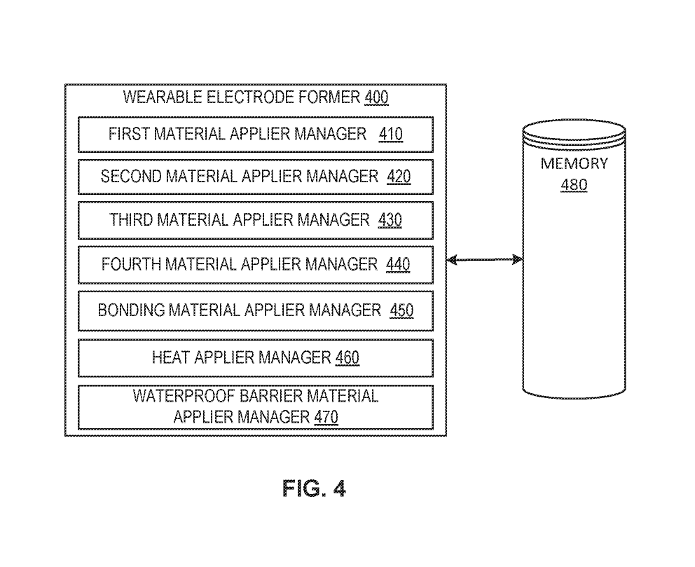

FIG. 4 is a block diagram of an example implementation of a wearable electrode former 400 to implement the example acts of fabrication of FIGS. 3A-3H to form the example wearables of FIGS. 1-2 in accordance with teachings of this disclosure.

The wearable electrode former 400 may be implemented by, for example, software existing on a processor within, for instance, a processing platform as discussed below. The example wearable electrode former 400 includes an example first material applier manager 410, an example second material applier manager 420, an example third material applier manager 430, an example fourth material applier manager 440, an example bonding material applier manager 450, an example heat applier manager 460 and an example waterproof barrier material applier manager 470.

In some examples, the example wearable electrode former 400 communicates with an example database 480 including process parameters (e.g., setpoints, timers, etc.) for the example first material applier manager 410, the example second material applier manager 420, the example third material applier manager 430, the example fourth material applier manager 440, the example bonding material applier manager 450, the example heat applier manager 460 and/or the example waterproof barrier material applier manager 470. In some examples, process parameters (e.g., setpoints, timers, etc.) are local to (e.g., within a local memory, such as an electrically erasable programmable read-only memory (EEPROM)) the example first material applier manager 410, the example second material applier manager 420, the example third material applier manager 430, the example fourth material applier manager 440, the example bonding material applier manager 450, the example heat applier manager 460 and/or the example waterproof barrier material applier manager 470.

The example first material applier manager 410 is to move the first material 300 from a first material store (e.g., a roll of the first material 300, a dispenser of pre-cut blanks of the first material 300, etc.) to a position and state (e.g., following cutting or punching of a portion of the roll of the first material 300, etc.) at which it may be worked upon as a base layer of a workpiece to form an example wearable electrode 120, 220 (FIGS. 1-2). The example first material applier manager 410 may control, for example, a conveyor, a material feed mechanism, a robot (e.g., a mechanical arm, mechanical fingers, pneumatic grippers, electric grippers, etc.), a vacuum cup pick and place device, etc., with associated sensors, to facilitate picking and/or positioning of the first material 300.

The example second material applier manager 420 is to move the second material 315 from a second material store (e.g., a roll of the second material 315, a dispenser of pre-cut blanks of the second material 315, a container of a second material 315 that is sprayable, etc.) to a position and state (e.g., following cutting or punching of a portion of the roll of the second material 315, etc.) at which it may be positioned on the workpiece including the first material 300, such as is shown in the example of FIG. 3D. The example second material applier manager 420 may control, for example, a conveyor, a material feed mechanism, a robot (e.g., a mechanical arm, mechanical fingers, pneumatic grippers, electric grippers, etc.), a vacuum cup pick and place device, etc., with associated sensors, to facilitate picking and/or positioning of the second material 315 on the workpiece (e.g., positioned on the first material 300, etc.).

The example third material applier manager 430 is to move the third material 325 from a third material store (e.g., a roll of the third material 325, a dispenser of pre-cut blanks of the third material 325, a container of a third material 325 that is sprayable, etc.) to a position and state (e.g., following cutting or punching of a portion of the roll of the third material 325, etc.) at which it may be positioned on the workpiece including the first material 300 and the second material 315, such as shown in the example of FIG. 3F. The example third material applier manager 430 may control, for example, a conveyor, a material feed mechanism, a robot (e.g., a mechanical arm, mechanical fingers, pneumatic grippers, electric grippers, etc.), a vacuum cup pick and place device, etc., with associated sensors, to facilitate picking and/or positioning of the third material 325 on the workpiece (e.g., positioned on the second material 315, etc.).

The example fourth material applier manager 440 is to move the fourth material 335 from a fourth material store (e.g., a roll of the fourth material 335, a dispenser of pre-cut blanks of the fourth material 335, etc.) to a position and state (e.g., following cutting or punching of a portion of the roll of the fourth material 335, etc.) at which it may be positioned on the workpiece including the first material 300, the second material 315, and the third material 325, such as shown in the example of FIG. 3H. The example fourth material applier manager 440 may control, for example, a conveyor, a material feed mechanism, a robot (e.g., a mechanical arm, mechanical fingers, pneumatic grippers, electric grippers, etc.), a vacuum cup pick and place device, etc., with associated sensors, to facilitate picking and/or positioning of the fourth material 335 on the workpiece (e.g., positioned on the second material 315, etc.).

The example bonding material applier manager 450 is to move a bonding material from one or more bonding material stores (e.g., a roll of a first bonding material 310, a container of a second bonding material 320 including a sprayable liquid, etc.) and to position the selected bonding material on the workpiece at one or more stages of construction of the example wearable electrode 120, 220. For example, in the example of FIGS. 3A-3H, the example bonding material applier manager 450 positions a first bonding material 310 on the waterproof barrier material 305 (FIG. 3C), positions a second bonding material 320 on the second material 315 (FIG. 3E), and positions a third bonding material 330 on the third material 325 (FIG. 3G). The first bonding material 310, the second bonding material 320 and/or the third bonding material 330 may be the same material, or may be different materials. The example fourth material applier manager 440 may control, for example, a conveyor, a material feed mechanism, a robot (e.g., a mechanical arm, mechanical fingers, pneumatic grippers, electric grippers, etc.), a vacuum cup pick and place device, etc., with associated sensors, to facilitate picking and/or positioning of the first bonding material 310, the second bonding material 320 and/or the third bonding material 330 on the workpiece (e.g., positioned on the second material 315 as shown in FIG. 3E). As noted, in some examples, the example first bonding material 310, the example second bonding material 320 and/or the example third bonding material 330 are omitted in the acts shown in FIGS. 3A-3H.

The example heat applier manager 460 is to apply heat, via a heating device (e.g., a heat press or mold, a flame, a hot wedge, a hot air welder, etc.), alone or in combination with pressure, to bond one or more materials. The example heat applier manager 460 is to cause a heating system to apply heat to any of the example first material 300, the example waterproof barrier material 305, the example first bonding material 310, the example second material 315, the example third material 325, the example third bonding material 330, the example fourth material 335 and/or combinations thereof at a predetermined temperature and for a predetermined time appropriate to the bonding material(s) selected to bond the one or more materials of the wearable electrode 120, 220.

The example waterproof barrier material applier manager 470 is to move the waterproof barrier material 305, where used, from a waterproof barrier material store (e.g., a roll of the waterproof barrier material 305, a dispenser of pre-cut blanks of the waterproof barrier material 305, a container of a waterproof barrier material 305 in the form of a sprayable liquid, etc.) to a position at which it may be positioned on the first material 300, such as is shown by way of example in FIG. 3B. The example waterproof barrier material applier manager 470 may control, for example, a conveyor, a material feed mechanism, a robot (e.g., a mechanical arm, mechanical fingers, pneumatic grippers, electric grippers, etc.), a vacuum cup pick and place device, a spray applicator, etc., with associated sensors, to facilitate picking, placing and/or application of the waterproof barrier material 305 to the workpiece including the first material 300.

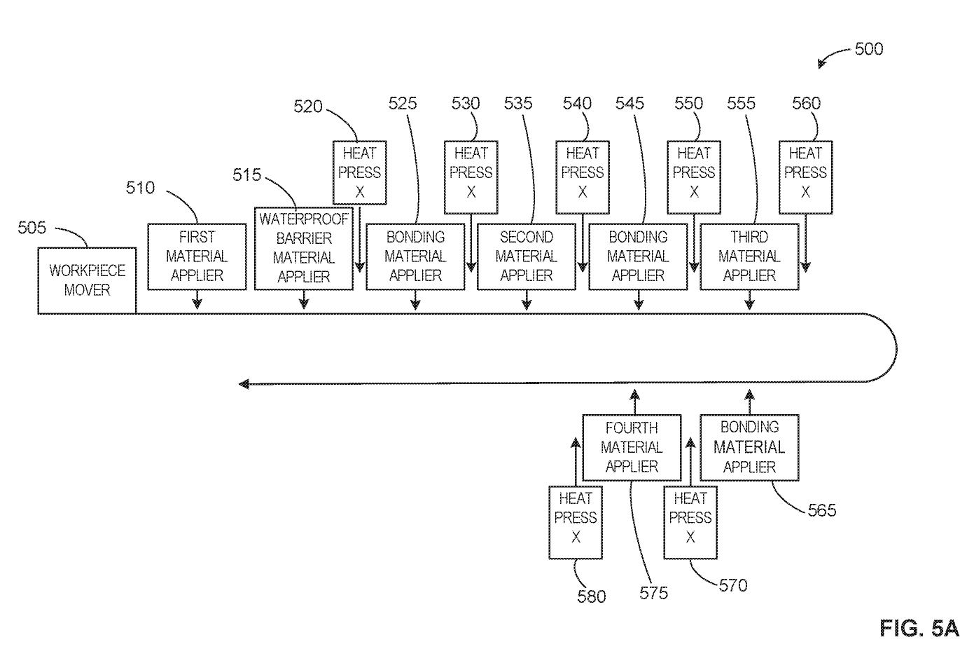

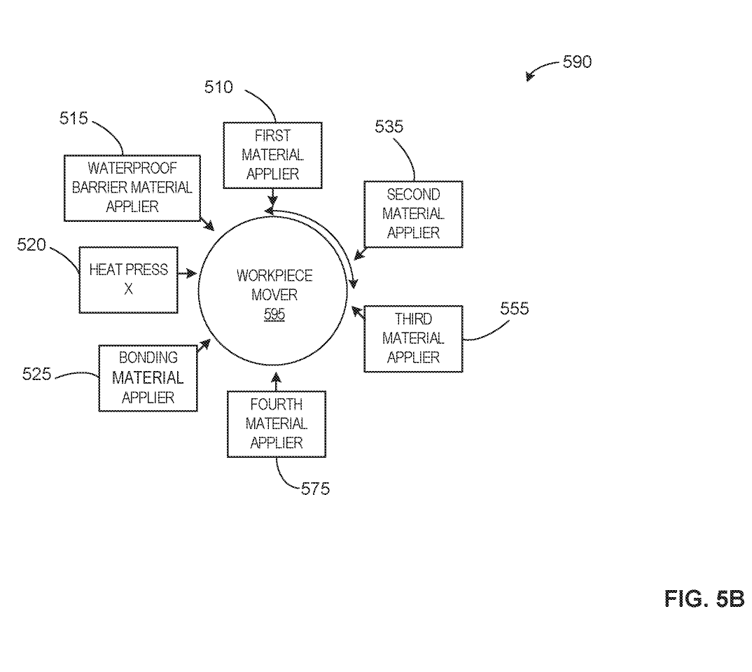

While example manners of constructing an example wearable electrode 120, 220 utilizing the example wearable electrode former 400 are set forth in FIGS. 5A-5B, other manners of fabrication may be used to fabricate the example wearable electrodes 120, 220. For instance, a plurality of layers of materials (e.g., the example first material 300, the example waterproof barrier material 305, the example first bonding material 310, the example second material 315, the example third material 325, the example third bonding material 330, and the example fourth material 335, etc.) may be simultaneously formed in sheets, bonded together by application of pressure and/or heat, and then cut or punched into desired shapes.

Example manners of constructing an example wearable electrode 120, 220 utilizing the example wearable electrode former 400 are set forth in FIGS. 5A-5B. The construction of the example wearable electrode 120, 220 and/or wearables 100, 200 include example wearable electrode(s) 120, 220, may be automated, semi-automated or manual. FIG. 5A shows an example U-shaped assembly line, whereas FIG. 5B shows an example rotary assembly line. Other types of assembly lines (e.g., straight, etc.) could alternatively be used.

FIG. 5A shows an electrode fabrication system 500 including an example workpiece mover 505, such as a conveyor belt or other conveyor system (e.g., lineshaft roller, etc.) to transport the workpiece from station to station. An example first material applier 510 operates via the example first material applier manager 410 to move the first material 300 from a first material store to a position on the example workpiece mover 505.

The example workpiece mover 505 moves the workpiece shown in FIG. 3A to the next station, the example waterproof barrier material applier 515. The example waterproof barrier material applier 515, via the example waterproof barrier material applier manager 470, obtains a waterproof barrier material 305 from a waterproof barrier material store and positions the waterproof barrier material 305 on the first material 300, such as is shown by way of example in FIG. 3B.

The example workpiece mover 505 then moves the workpiece shown in FIG. 3B to the next station, the example heat press or mold 520 (hereinafter “heat press”). The example heat press 520, via the example heat applier manager 460, applies heat and pressure to bond the workpiece including the first material 300 and the example waterproof barrier material 305.

The example workpiece mover 505 then moves the workpiece shown in FIG. 3C to the example bonding material applier 525. The example bonding material applier 525, via the example bonding material applier manager 450, obtains a first bonding material 310 from a first bonding material store and positions the first bonding material 310 in contact with the waterproof barrier material 305, as shown in the example of FIG. 3C.

The example workpiece mover 505 then moves the workpiece shown in FIG. 3C to the example heat press 530. The example heat press 530, via the example heat applier manager 460, applies heat and pressure to bond the workpiece including the first material 300, the example waterproof barrier material 305 and the example first bonding material 310.

The example workpiece mover 505 then moves the workpiece shown in FIG. 3C to the next station, the example second material applier 535. The example second material applier 535, via the example second material applier manager 420, positions the second material 315 on the workpiece including the first material 300, such as is shown in the example of FIG. 3D (e.g., in contact with the example first bonding material 310).

The example workpiece mover 505 then moves the workpiece shown in FIG. 3D to the example heat press 540. The example heat press 540, via the example heat applier manager 460, applies heat and pressure to bond the workpiece including the first material 300, the example waterproof barrier material 305, the example first bonding material 310, and the example second material 315.

The example workpiece mover 505 then moves the workpiece shown in FIG. 3C to the example bonding material applier 545. The example bonding material applier 545, via the example bonding material applier manager 450, obtains a second bonding material 320 from a second bonding material store and positions the example second bonding material 320 in contact with the example second material 315, as shown in the example of FIG. 3E.

The example workpiece mover 505 then moves the workpiece shown in FIG. 3E to the example heat press 550. The example heat press 540, via the example heat applier manager 460, applies heat and pressure to bond the workpiece including the first material 300, the example waterproof barrier material 305, the example first bonding material 310, the example second material 315, and the example second bonding material 320.

The example workpiece mover 505 then moves the workpiece shown in FIG. 3C to the example third material applier 555. The example third material applier 555, via the example third material applier manager 430, positions the example third material 325 on the workpiece including the first material 300 and the second material 315, such as is shown in the example of FIG. 3F (e.g., in contact with the example second bonding material 320).

The example workpiece mover 505 then moves the workpiece shown in FIG. 3F to the example heat press 560. The example heat press 560, via the example heat applier manager 460, applies heat and pressure to bond the workpiece including the first material 300, the example waterproof barrier material 305, the example first bonding material 310, the example second material 315, the example second bonding material 320 and the example third material 325.

The example workpiece mover 505 then moves the workpiece shown in FIG. 3F to the example bonding material applier 565. The example bonding material applier 565, via the example bonding material applier manager 450, obtains a third bonding material 330 from a third bonding material store and positions the example third bonding material 330 in contact with the example third material 325, as shown in the example of FIG. 3G.

The example workpiece mover 505 then moves the workpiece shown in FIG. 3G to the example heat press 570. The example heat press 570, via the example heat applier manager 460, applies heat and pressure to bond the workpiece including the first material 300, the example waterproof barrier material 305, the example first bonding material 310, the example second material 315, the example second bonding material 320, the example third material 325 and the example third bonding material 330.

The example workpiece mover 505 then moves the workpiece shown in FIG. 3G to the example fourth material applier 575. The example fourth material applier 575, via the example fourth material applier manager 440, positions the example fourth material 335 on the workpiece including the example first material 300, the example second material 315 and the example third material 325, as shown in the example of FIG. 3H (e.g., in contact with the example third bonding material 330).

The example workpiece mover 505 then moves the workpiece shown in FIG. 3H to the example heat press 580. The example heat press 580, via the example heat applier manager 460, applies heat and pressure to bond the workpiece including the first material 300, the example waterproof barrier material 305, the example first bonding material 310, the example second material 315, the example second bonding material 320, the example third material 325, the example third bonding material 330 and the example fourth material 335.

FIG. 5B is similar to FIG. 5A, except in that the electrode fabrication system 590 of FIG. 5B uses a rotary workpiece mover 595 having a smaller footprint than the U-shaped workpiece mover 505 of FIG. 5A. Additionally, the example rotary workpiece mover 595 is able to use an example heat press 520 to perform all acts of heating of the workpiece and an example bonding material applier 525 to perform all acts of application of a bonding material to the workpiece.

While an example manner of implementing the example wearable electrode former 400 is set forth in FIG. 4, one or more of the elements, processes and/or devices illustrated in FIG. 4 may be combined, divided, re-arranged, omitted, eliminated and/or implemented in any other way. For example, the example first material applier manager 410, the example second material applier manager 420, the example third material applier manager 430, the example fourth material applier manager 440, the example bonding material applier manager 450, the example heat applier manager 460 and/or the example waterproof barrier material applier manager 470 may be implemented by hardware, software, firmware and/or any combination of hardware, software and/or firmware. Thus, for example, any or all of the example first material applier manager 410, the example second material applier manager 420, the example third material applier manager 430, the example fourth material applier manager 440, the example bonding material applier manager 450, the example heat applier manager 460 and/or the example waterproof barrier material applier manager 470 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)).

Further, while an example manner of implementing the example wearable electrode former 400 is set forth in FIG. 4, one or more additional elements may be combined with the processes and/or devices illustrated in FIG. 4 or with the processes and/or devices otherwise described herein. For instance, the example electrode fabrication systems 500, 590 of FIGS. 5A-5B could include a local device 130, 240 applier to place a local device 130, 240 in electrical connection with the example electrical pathways 140, 250.

When reading any of the apparatus or system claims of this patent to cover a purely software and/or firmware implementation, at least one of the example first material applier manager 410, the example second material applier manager 420, the example third material applier manager 430, the example fourth material applier manager 440, the example bonding material applier manager 450, the example heat applier manager 460 and/or the example waterproof barrier material applier manager 470 are hereby expressly defined to include a non-transitory computer-readable storage device or storage disk such as a memory, a digital versatile disk (DVD), a compact disk (CD), a Blu-ray disk, a flash memory, etc. storing the software and/or firmware. Further still, the example first material applier manager 410, the example second material applier manager 420, the example third material applier manager 430, the example fourth material applier manager 440, the example bonding material applier manager 450, the example heat applier manager 460 and/or the example waterproof barrier material applier manager 470 may include one or more elements, processes and/or devices in addition to, or instead of, those illustrated in FIGS. 1-3H, for example, and/or may include more than one of any or all of the illustrated elements, processes and devices.

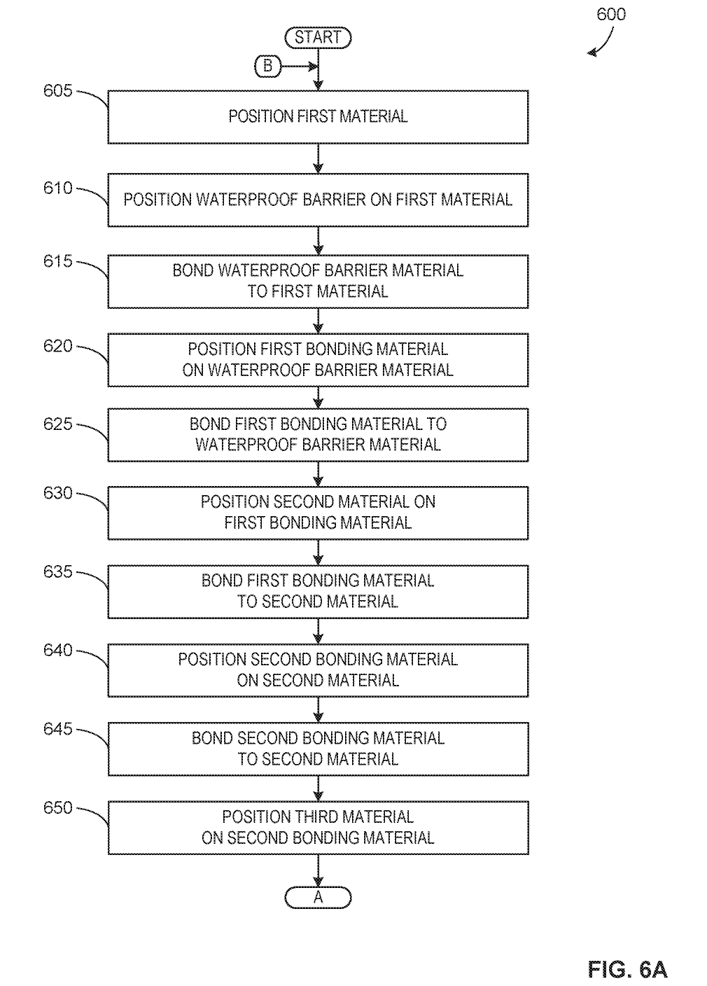

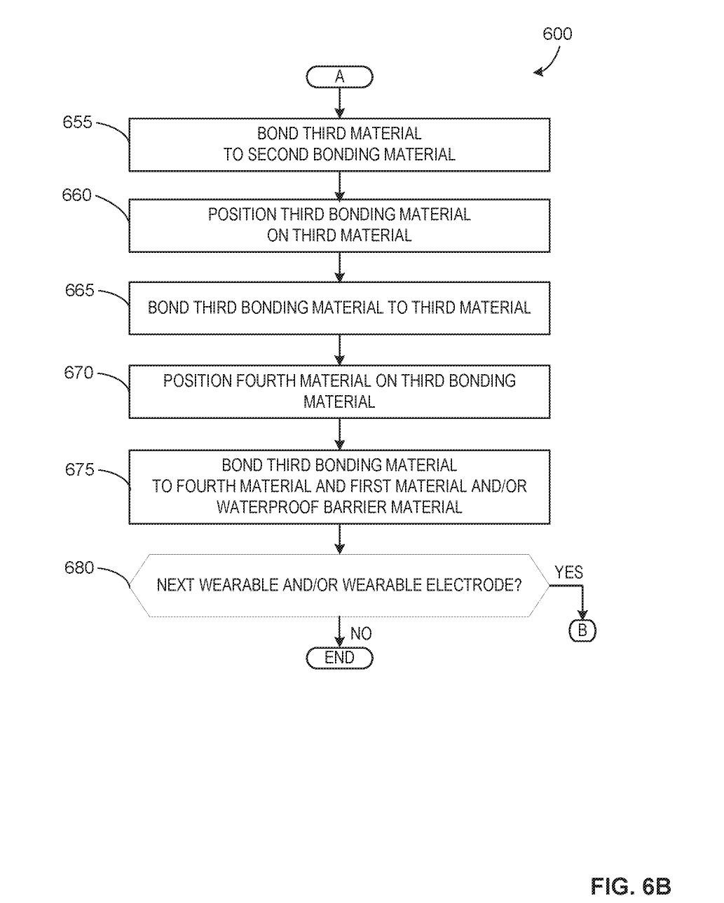

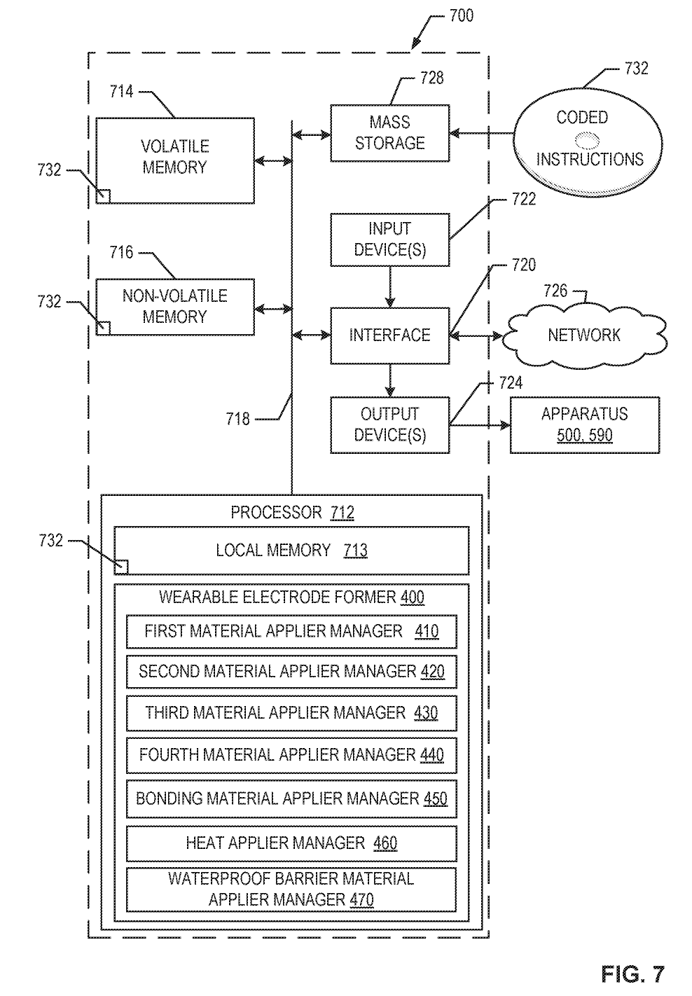

An example flowchart representing example machine readable instructions for implementing the example wearable electrode former 400 of FIG. 4, using the example electrode fabrication systems 500, 590 of FIGS. 5A-5B, is shown in FIGS. 6A-6B. In the example of FIGS. 6A-6B, the machine-readable instructions are a program for execution by one or more processors, such as the example processor platform 700 discussed below in connection with FIG. 7. The program may be embodied in software stored on a non-transitory computer-readable storage medium such as a CD-ROM, a floppy disk, a hard drive, a digital versatile disk (DVD), a Blu-ray disk, a cloud-based server memory, a remote computer memory, or a memory associated with the example processor 712, but the entire program and/or parts thereof could alternatively be executed by a device other than the example processor 712 and/or embodied in firmware or dedicated hardware. Further, although the example program is described with reference to the flowcharts illustrated in FIGS. 6A-6B, many other methods of implementing the example wearable electrode former 400 may alternatively be used. For example, the order of execution of the blocks in FIGS. 6A-6B may be changed, and/or some of the blocks described may be changed, eliminated, and/or combined.

As mentioned above, the example machine readable instructions shown in FIGS. 6A-6B for implementing the example wearable electrode former 400 disclosed herein, may be implemented using coded instructions (e.g., computer and/or machine readable instructions) stored on a non-transitory computer and/or machine readable medium, wherever located, such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used (e.g., as the transition term in a preamble of a claim), it is open-ended in the same manner as the term “comprising” is open ended.

The example program 600 of FIGS. 6A-6B is discussed below in the context of the example wearable electrode former 400 of FIG. 4 and the example electrode fabrication system 590 of FIG. 5B. The example program 600 of FIGS. 6A-6B begins at block 605 where the example wearable electrode former 400 causes the example first material applier 510, via the example first material applier manager 410, to position the example first material 300 in a work space where it can be worked upon to construct a wearable electrode 120, 220 thereof. Control then passes to block 610.

At block 610, the example wearable electrode former 400 causes the waterproof barrier material applier 515, via the example waterproof barrier material applier manager 470, to position waterproof barrier material 305 on the first material 300, such as shown in FIG. 3B. Control then passes to block 615.

At block 615, the example wearable electrode former 400 causes the heat press 520, via the example heat applier manager 460, to bond the waterproof barrier material 305 to the first material 300. Control then passes to block 620.

At block 620, the example wearable electrode former 400 causes the bonding material applicator 525, via the example heat applier manager 460, to position the first bonding material 310 on the waterproof barrier material 305. Control then passes to block 625.

At block 625, the example wearable electrode former 400 causes the heat press 520, via the example bonding material applier manager 450, to bond the first bonding material 310 to the waterproof barrier material 305. Control then passes to block 630.

At block 630, the example wearable electrode former 400 causes the second material applier 535, via the example second material applier manager 420, to position the second material 315 on the first material 300 (e.g., in contact with the first bonding material 310), such as shown in FIG. 3D. Control then passes to block 635.

At block 635, the example wearable electrode former 400 causes the heat press 520, via the example heat applier manager 460, to bond the second material 315 to the first bonding material 310. Control then passes to block 640.

At block 640, the example wearable electrode former 400 causes the bonding material applicator 525, via the example bonding material applier manager 450, to position the second bonding material 320 on the second material 315. Control then passes to block 645.

At block 645, the example wearable electrode former 400 causes the heat press 520, via the example heat applier manager 460, to bond the second bonding material 320 to the second material 315. Control then passes to block 650.

At block 650, the example wearable electrode former 400 causes the third material applier 555, via the example third material applier manager 430, to position the third material 325 on the second material 315 (e.g., in contact with the second bonding material 320), such as shown in FIG. 3F. Control then passes to block 655 in FIG. 6B.

At block 655, the example wearable electrode former 400 causes the heat press 520, via the example heat applier manager 460, to bond the third material 325 to the second bonding material 320. Control then passes to block 660.

At block 660, the example wearable electrode former 400 causes the bonding material applicator 525, via the example bonding material applier manager 450, to position the third bonding material 330 on the third material 325. Control then passes to block 665.

At block 665, the example wearable electrode former 400 causes the heat press 520, via the example heat applier manager 460, to bond the third bonding material 330 to the third material 325. Control then passes to block 670.

At block 670, the example wearable electrode former 400 causes the fourth material applier 575, via the example fourth material applier manager 440, to position the fourth material 335 on the third material 325 (e.g., in contact with the third bonding material 330), such as shown in FIG. 3H. Control then passes to block 675.

At block 675, the example wearable electrode former 400 causes the heat press 520, via the example heat applier manager 460, to bond the third bonding material 330 to the fourth material 335 and to bond the fourth material 335 to the first material 300 and/or the waterproof barrier material 305. Control then passes to block 680.

At block 680, the example wearable electrode former 400 determines whether additional wearables 100, 200 and/or wearable electrodes 120, 220 are to be processed. For example, where a plurality of wearables 100, 200 are to be produced in a batch and the production is not yet complete, block 680 is “YES” and control passes to block 605 for the construction of a next wearable 100, 200 in the batch. If the production of the batch is complete and the result of block 680 is “NO,” the process ends. In another example, where wearable electrodes 120, 220 are produced for a wearable 100, 200 and the wearable electrodes 120, 220 are produced in multiple runs through one or more electrode fabrication systems 500, 590 (e.g., wearable electrodes 120, 220 are formed in different portions of the wearable 100, 200, having different material requirements or different processing requirements, etc.), block 680 determines if additional wearable electrodes 120, 220 are to be formed. If block 680 is “YES,” control passes to block 605 and if block 680 is “NO,” the process ends.

As noted above, FIG. 7 is a block diagram of an example processor platform 700 capable of executing the example instructions of FIGS. 6A-6B to implement the example wearable electrode former 400 of FIG. 4 and/or the example electrode fabrication systems 500, 590 of FIGS. 5A-5B to form the example wearable electrodes 120, 220 of FIGS. 1-3H. The processor platform 700 may be implemented by a server, a desktop computer, a laptop computer, a terminal, a mobile device (e.g., a tablet computer, such as an iPad™), a dedicated device, or any other type of computing device.

The processor platform 700 of the illustrated example includes a processor 712. The processor 712 of the illustrated example is hardware. For example, the processor 712 can be implemented by integrated circuits, logic circuits, microprocessors or controllers from any desired family or manufacturer. In the example of FIG. 7, the processor 712 implements the wearable electrode former 400. As such, it implements the example first material applier manager 410, the example second material applier manager 420, the example third material applier manager 430, the example fourth material applier manager 440, the example bonding material applier manager 450, the example heat applier manager 460 and/or the example waterproof barrier material applier manager 470.

The processor 712 of the illustrated example includes a local memory 713 (e.g., a cache). The processor 712 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 716 may be implemented by flash memory and/or any other desired type of memory device. Access to the main memory (e.g., 714, 716) is controlled by a memory controller.

The processor platform 700 of the illustrated example also includes an interface circuit 720. The interface circuit 720 may be implemented by any type of interface standard, such as an Ethernet interface, a universal serial bus (USB), and/or a PCI express interface.

In the illustrated example, input device(s) 722 are connected to the interface circuit 720. The input device(s) 722 permit(s) a user to enter data and commands into the processor 712. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 724 are also connected to the interface circuit 720 of the illustrated example. The output devices 724 can be implemented, for example, by display devices (e.g., a light emitting diode (LED), an organic light emitting diode (OLED), a liquid crystal display, a cathode ray tube display (CRT), a touchscreen, a tactile output device, a printer, speakers, etc.). In some examples, the interface circuit 720 includes a graphics driver card, a graphics driver chip or a graphics driver processor.

The interface circuit 720 of the illustrated example also includes a communication device such as a transmitter, a receiver, a transceiver, a modem and/or network interface card to facilitate exchange of data with external machines (e.g., computing devices of any kind) via a network 726 (e.g., an Ethernet connection, a digital subscriber line (DSL), a telephone line, coaxial cable, a cellular telephone system, etc.).

The processor platform 700 of the illustrated example also includes mass storage devices 728 for storing software and/or data. Examples of such mass storage devices 728 include floppy disk drives, hard drive disks, compact disk drives, Blu-ray disk drives, RAID systems, and digital versatile disk (DVD) drives.

The coded instructions 732 of FIG. 7, represented generally in FIGS. 6A-6B, may be stored in the mass storage device 728, in the volatile memory 714, in the non-volatile memory 716, and/or on a removable tangible computer readable storage medium such as a CD, DVD or solid-state memory device.

Example 1 is a wearable electrode, including a first layer of a first material, a second material positioned on the first material, the second material having a first compressive strength, a third material positioned on the second material, the third material having a second compressive strength different than the first compressive strength and a fourth material including a conductive element positioned on the third material, positioned around the second material, and joined to the first material.

Example 2 includes the wearable electrode as defined in Example 1, wherein the first material includes a waterproof barrier.

Example 3 includes the wearable electrode as defined in Example 1, further including a waterproof barrier positioned between the first material and the second material, wherein the fourth material is joined to the waterproof barrier.

Example 4 includes the wearable electrode as defined in any one of Examples 1-3, wherein the first material is bonded to the second material, the second material is bonded to the third material, or the third material is bonded to the fourth material.

Example 5 includes the wearable electrode as defined in any one of Examples 1-3, wherein the second material is in contact with the first material, the third material is in contact with the second material, or the fourth material is in contact with the third material.

Example 6 includes the wearable electrode as defined in any one of Examples 1-3, wherein the first compressive strength is between about 10-40 PSI and wherein the second compressive strength is between about 1-10 PSI.

Example 7 includes the wearable electrode as defined in Example 6, wherein the second material is a first foam material and the third material is a second foam material.

Example 8 includes the wearable electrode as defined in Example 7, wherein the first material includes a fabric and the fourth material includes a conductive fabric.

Example 9 includes the wearable electrode as defined in Example 8, wherein the fourth material is a skin-facing inner layer of a garment, an electronic textile, or a medical device.

Example 10 is a method of forming a wearable electrode, including positioning a second material having a first compressive strength on a first material, positioning a third material having a second compressive strength on the second material, positioning a fourth material including a conductive element on the third material and around the second material and sealing the fourth material around the second material and the third material to form the wearable electrode on the first material.

Example 11 includes the method as defined in Example 10, wherein the second compressive strength is lower than the first compressive strength.

Example 12 includes the method as defined in Example 10 or Example 11, wherein the sealing includes at least one of heat sealing, adhesive bonding, ultrasonic welding, radio frequency welding, curing, sewing or riveting.

Example 13 includes the method of Example 12, wherein the fourth material is a skin-facing inner layer of a garment, an electronic textile, or a medical device.

Example 14 includes the method of Example 12, wherein the first material, the second material, the third material and the fourth material to form the wearable electrode are flexible.

Example 15 includes the method of Example 14, wherein the first material includes a fabric and the fourth material includes a conductive fabric.

Example 16 includes the method of Example 15, wherein at least one of the second material or the third material includes a foam.

Example 17 includes the method of any one of Examples 10-12, wherein the first compressive strength is between about 10-40 PSI, and wherein the second compressive strength is between about 1-10 PSI.

Example 18 includes the method of any one of Examples 10-12, wherein the second material is in contact with the first material, the third material is in contact with the second material, or the fourth material is in contact with the third material.

Example 19 includes the method of any one of Examples 10-12, and further includes applying a bonding material between one or more of the first material and the second material, the second material and the third material, and the third material and the fourth material and bonding the one or more of the first material and the second material, the second material and the third material, and the third material and the fourth material.

Example 20 includes the method of Example 19, wherein the bonding includes heating the one or more of the first material and the second material, the second material and the third material, and the third material and the fourth material having the bonding material positioned therebetween to a temperature between about 125° C.-175° C. for between about 5-45 seconds.

Example 21 is a wearable including a wearable electrode, the wearable electrode including a first material, a second material having a first compressive strength positioned on the first material, a third material having a second compressive strength different than the first compressive strength positioned on the second material, and a fourth material overlaying the first material, second material and third material, the fourth material being joined to the first material. The wearable also includes a conductive element extending from a surface of the fourth material to the first material and a local device operatively coupled to the wearable electrode.

Example 22 includes the wearable of Example 21, wherein the second material is a first foam material and the third material is a second foam material.

Example 23 includes the wearable of Example 21, wherein the wearable electrode includes a waterproof barrier positioned between the first material and the second material, and wherein the fourth material is joined to the waterproof barrier.

Example 24 includes the wearable of any one of Examples 21-23, wherein the first material is bonded to the second material, the second material is bonded to the third material, or the third material is bonded to the fourth material.

Example 25 includes the wearable of any one of Examples 21-23, wherein the second material is in contact with the first material, the third material is in contact with the second material, or the fourth material is in contact with the third material.

Example 26 includes the wearable of any one of Examples 21-23, wherein the first compressive strength is between about 10-40 PSI and wherein the second compressive strength is between about 1-10 PSI.

Example 27 includes the wearable of Example 26, wherein the first material includes a waterproof barrier.

Example 28 includes the wearable of any one of Examples 21-23, wherein the first material includes a fabric and the fourth material includes a conductive fabric.

Example 29 includes the wearable of any one of Examples 21-23, wherein the wearable includes a garment, an electronic textile, a wearable material, a bra, a shirt, a pant, shorts, a sock, a band, a compression garment, or a medical device.

Example 30 includes the wearable of Example 21, wherein the wearable includes a plurality of wearable electrodes.

Example 31 includes the wearable of Example 30, wherein plurality of wearable electrodes are arranged in a pattern on the wearable.

Example 32 includes the wearable of Example 30, wherein the plurality of wearable electrodes includes a first set of wearable electrodes and a second set of wearable electrodes, and wherein the first set of wearable electrodes has a first compressive strength or a second compressive strength that is different than a first compressive strength or a second compressive strength of the second set of wearable electrodes.

Example 33 includes the wearable of any one of Examples 30-32, wherein the wearable electrodes are arranged about the wearable to provide heart rate data, respiration rate data or breathing depth data from a user of the wearable to the local device.

Example 34 includes the wearable of any one of Examples 30-32, wherein the wearable electrodes are operatively connected to the local device via a hardwired connection or via a wireless connection.

Example 35 includes the wearable of any one of Examples 30-32, wherein the local device includes at least one of a 3-axis accelerometer, a clock, a GPS device, a transceiver, a thermometer or a bioimpedance sensor.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A wearable electrode, comprising:
a first layer of a first material;
a second material positioned on the first material, the second material having a first compressive strength;
a third material positioned on the second material, the third material having a second compressive strength different than the first compressive strength; and
a fourth material including a conductive element positioned on the third material, positioned around the second material, and joined to the first material.

2. The wearable electrode of claim 1, wherein the first material includes a waterproof barrier.

3. The wearable electrode of claim 1, further including a waterproof barrier positioned between the first material and the second material, wherein the fourth material is joined to the waterproof barrier.

4. The wearable electrode of claim 1, wherein the first material is bonded to the second material, the second material is bonded to the third material, or the third material is bonded to the fourth material.

5. The wearable electrode of claim 1, wherein the second material is in contact with the first material, the third material is in contact with the second material, or the fourth material is in contact with the third material.

6. The wearable electrode of claim 1, wherein the first compressive strength is between about 10-40 PSI and wherein the second compressive strength is between about 1-10 PSI.

7. The wearable electrode of claim 6, wherein the second material is a first foam material and the third material is a second foam material.

8. The wearable electrode of claim 7, wherein the first material includes a fabric and the fourth material includes a conductive fabric.

9. The wearable electrode of claim 8, wherein the fourth material is a skin-facing inner layer of a garment, an electronic textile, or a medical device.

10. A method of forming a wearable electrode, comprising:
positioning a second material having a first compressive strength on a first material;
positioning a third material having a second compressive strength on the second material;
positioning a fourth material including a conductive element on the third material and around the second material; and
sealing the fourth material around the second material and the third material to form the wearable electrode on the first material.

11. The method of claim 10, wherein the second compressive strength is lower than the first compressive strength.

12. The method of claim 11, wherein the sealing includes at least one of heat sealing, adhesive bonding, ultrasonic welding, radio frequency welding, curing, sewing or riveting.

13. The method of claim 12, wherein the first material, the second material, the third material and the fourth material to form the wearable electrode are flexible.

14. The method of claim 13, wherein the first material includes a fabric and the fourth material includes a conductive fabric.

15. The method of claim 14, wherein at least one of the second material or the third material includes a foam.

16. The method of claim 15, wherein the first compressive strength is between about 10-40 PSI, and wherein the second compressive strength is between about 1-10 PSI.

17. The method of claim 12, further including applying a bonding material between one or more of the first material and the second material, the second material and the third material, and the third material and the fourth material and bonding the one or more of the first material and the second material, the second material and the third material, and the third material and the fourth material.

18. The method of claim 17, wherein the bonding includes heating the one or more of the first material and the second material, the second material and the third material, and the third material and the fourth material having the bonding material positioned therebetween to a temperature between about 125° C.-175° C. for between about 5-45 seconds.

19. A wearable, comprising:
a wearable electrode, the wearable electrode including a first material, a second material having a first compressive strength positioned on the first material, a third material having a second compressive strength different than the first compressive strength positioned on the second material, and a fourth material overlaying the first material, second material and third material, the fourth material being joined to the first material;
a conductive element extending from a surface of the fourth material to the first material; and
a local device operatively coupled to the wearable electrode.

20. The wearable of claim 19, wherein the second material is a first foam material and the third material is a second foam material.
